(19) United States
(12) Patent Application Publication
(10) Pub. No.: US 2025/0302546 A1
(43) Pub. Date: Oct. 2, 2025

(54) SYSTEMS AND METHODS FOR RECALLING TRACKING INFORMATION VIA APPLIED LANDMARKS

(71) Applicants: Smith & Nephew, Inc., Memphis, TN (US); Smith & Nephew Orthopaedics AG, Zug (CH)

(72) Inventors: Samuel C. DUMPE, Sewickley, PA (US); Branislav JARAMAZ, Pittsburgh, PA (US); Edward DAVIS, Worcestershire (GB); Thorston M. SEYLER, Durham, NC (US); Parker HILL, Pittsburgh, PA (US)

(21) Appl. No.: 19/090,677

(22) Filed: Mar. 26, 2025

Related U.S. Application Data
(60) Provisional application No. 63/572,488, filed on Apr. 1, 2024.

(51) Int. Cl. A61B 34/20; A61B 34/10; A61B 90/00

(52) U.S. Cl. CPC A61B 34/20; A61B 34/10; A61B 90/39; A61B 2034/105; A61B 2034/2055; A61B 2034/2068; A61B 2090/3916

(57) ABSTRACT

Systems and methods for tracking in a surgical procedure are disclosed herein. A method may include receiving at least one of an image and a statistical shape model of a patient's anatomy. A tracking system may acquire a tracking marker location and register the patient's anatomy relative to the tracking marker. A rigid body may be affixed the patient's anatomy and registered relative to the tracking marker by capturing landmarks on the surface of the rigid body. A transformation matrix may be generated between the location of the rigid body and a location of the patient anatomy. The tracking marker may then be removed, with the location of the anatomy recalled by recapturing the locations of the landmarks and applying the transformation.

### CROSS-REFERENCE TO RELATED APPLICATIONS

This application claims the benefit of U.S. Provisional Application No. 63/572,488, filed Apr. 1, 2024, which is herein incorporated by reference.

### TECHNICAL FIELD

The present disclosure relates generally to methods, systems, and apparatuses related to optical tracking markers.

### BACKGROUND

In the past, navigated/robotic surgical systems have taken varied approaches to tracking the femur in a total hip arthroplasty (THA) procedure. Surgical systems may include detachable arrays, typically attached to a fixed baseplate near the greater trochanter. Alternatively, some surgical systems rely solely on landmarks collected relative to a tracker on the pelvis. In the past, detachable arrays have not provided the necessary stability to accurately track the position of the femur, particularly while accommodating the interference of nearby soft tissue. Obtaining access to attach an array may also be a challenge depending on the surgical approach. Tracking solely through landmarks may lack reliability as the amount of information available related to the femoral anatomy is limited.

Thus, a method of tracking is needed that provides the tracking reliability of an affixed tracking array without the tracking array interfering with the THA procedure.

### SUMMARY

In some embodiments, a tracking device includes a rigid plate comprising one or more affixation points configured to receive a screw for securing the rigid plate to patient anatomy; and at least three divots located on an outfacing surface of the rigid plate, wherein each wherein a location of divot is configured to be reliably captured using a point probe.

In some embodiments, the at least three divots are arranged on the outfacing surface in an at least partially asymmetrical configuration, such that an orientation of the rigid plate is unambiguous based on a location of the at least three divots.

In some embodiments, an electrosurgical device includes a housing; an electrically conductive femoral neck guide configured to a shape of a femoral neck, wherein the electrically conductive femoral neck guide is rigidly affixable to the housing; a control configured to apply a high current, low voltage AC waveform to the electrically conductive femoral neck guide; and a tracking marker rigidly affixable to the housing.

In some embodiments, the tracking marker includes a point probe, wherein the point probe is removable from the housing.

In some embodiments, the control is configured to apply the AC waveform in response to an input from a tracking system suggesting the electrically conductive femoral neck guide is placed according to a surgical plan.

In some embodiments, the control is configured to apply the AC waveform in further response to a manual input.

In some embodiments, a computer-implemented method includes receiving, by a processor, at least one of an image and a statistical shape model of a patient anatomy; acquiring, using a tracking system in communication with the processor, a tracking marker location of a tracking marker; acquiring, using the tracking system, a plurality of point probe locations of a trackable point probe, wherein for each of the plurality of point probe locations the trackable point probe is positioned relative to the patient anatomy; registering, by the processor, a first position and orientation of the patient anatomy relative to the tracking marker based on the tracking marker location and the plurality of point probe locations; acquiring, using the tracking system, landmark locations of at least three landmarks on a rigid, low-profile body configured to affix to the patient anatomy; registering, by the processor, a position and orientation of the rigid, low-profile body relative to the tracking marker location based on the tracking marker location and the landmark locations; generating, by the processor, a transformation matrix between the position and orientation of the rigid, low-profile body and the first position and orientation of the patient anatomy; acquiring, using the tracking system, updated landmark locations of the at least three landmarks, absent the tracking marker; and determining, by the processor, a second position and orientation of the patient anatomy based on the updated landmark locations and the transformation matrix.

In some embodiments, each of the at least three landmarks include a divot configured to be reliably captured by the trackable point probe.

In some embodiments, the at least three landmarks are arranged on a surface of the rigid, low-profile body in an at least partially asymmetrical configuration, such that an orientation of the rigid, low-profile body is unambiguous based on a location of the at least three landmarks.

In some embodiments, the method includes navigating, by the processor, a marking device configured to mark the patient anatomy for a cut between a first affixation point of the tracking marker to the patient anatomy and a second affixation point of the rigid, low-profile body to the patient anatomy.

In some embodiments, the cut is configured to remove a femoral head from the patient anatomy, and wherein the tracking marker is affixed to the femoral head.

In some embodiments, the tracking marker is affixed to the patient anatomy via a femoral head remover.

In some embodiments, the tracking system is an optical tracking system and the method includes acquiring, using the tracking system, the position and orientation of the rigid, low-profile body based on an optical detection of the landmark locations.

In some embodiments, a system includes a tracking system; a tracking marker configured to affix to a patient anatomy at a first affixation point; a trackable point probe; a rigid, low-profile body configured to affix to the patient anatomy at a second affixation point, wherein the rigid, low-profile body comprises at least three landmarks; a processor in communication with the tracking system; and a non-transitory, processor-readable storage medium. The non-transitory, processor-readable storage medium may include one or more programming instructions that, when executed, cause the processor to receive at least one of an image and a statistical shape model of the patient anatomy; acquire, using the tracking system, a tracking marker location of the tracking marker; acquire, using the tracking system, a plurality of point probe locations of the trackable point probe, wherein for each of the plurality of point probe locations the trackable point probe is positioned relative to the patient anatomy; register a first position and orientation of the patient anatomy relative to the tracking marker based on the tracking marker location and the plurality of point probe locations; acquire, using the tracking system, landmark locations of the at least three landmarks; register a position and orientation of the rigid, low-profile body relative to the tracking marker location based on the tracking marker location and the landmark locations; generate a transformation matrix between the position and orientation of the rigid, low-profile body and the first position and orientation of the patient anatomy; acquire, using the tracking system, updated landmark locations of the at least three landmarks, absent the tracking marker; and determine a second position and orientation of the patient anatomy based on the updated landmark locations and the transformation matrix.

In some embodiments, each of the at least three landmarks include a divot configured to be reliably captured by the trackable point probe.

In some embodiments, the at least three landmarks are arranged on a surface of the rigid, low-profile body in an at least partially asymmetrical configuration, such that an orientation of the rigid, low-profile body is unambiguous based on a location of the at least three landmarks.

In some embodiments, the system includes a marking device and the one or more programming instructions further cause the processor to navigate the marking device configured to mark the patient anatomy for a cut between a first affixation point of the tracking marker to the patient anatomy and a second affixation point of the rigid, low-profile body to the patient anatomy.

In some embodiments, the marking device is an electrosurgical device.

In some embodiments, the cut is configured to remove a femoral head from the patient anatomy, and wherein the tracking marker is affixed to the femoral head.

In some embodiments, the tracking marker is affixed to the patient anatomy via a femoral head remover.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts an operating theatre including an illustrative computer-assisted surgical system (CASS) in accordance with an embodiment.

FIG. 2A depicts illustrative control instructions that a surgical computer provides to other components of a CASS in accordance with an embodiment.

FIG. 2B depicts illustrative control instructions that components of a CASS provide to a surgical computer in accordance with an embodiment.

FIG. 2C depicts an illustrative implementation in which a surgical computer is connected to a surgical device in accordance with an embodiment.

FIG. 3 depicts a flow chart for a method of obtaining six degrees of freedom measurement data after a stable tracking marker has been removed from the anatomy in accordance with an embodiment.

FIG. 4 depicts a tracking marker affixed to the femoral head/neck junction in accordance with an embodiment.

FIG. 5 depicts an illustrative operation of registering landmarks of the anatomy using a point probe in accordance with an embodiment.

FIG. 6 illustrates a rigid body attached to the anatomy in accordance with an embodiment.

FIG. 7 depicts an illustrative operation of collecting registration data from a landmark using a point probe in accordance with an embodiment.

FIG. 8 depicts a cut plane for a femoral neck cut in accordance with an embodiment.

FIG. 9 depicts an illustrative operation of acquiring a snapshot measurement of the anatomy with a point probe in accordance with an embodiment.

FIG. 10 depicts an electrosurgical device configured to mark the bone at a proper resection level and angle in accordance with an embodiment.

FIG. 11 depicts the electrosurgical device interfaced with a point probe in accordance with an embodiment.

FIG. 12 illustrates a block diagram of an exemplary data processing system in which embodiments are implemented.

### DETAILED DESCRIPTION

This disclosure is not limited to the particular systems, devices and methods described, as these may vary. The terminology used in the description is for the purpose of describing the particular versions or embodiments only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

### Definitions

For the purposes of this disclosure, the term “implant” is used to refer to a prosthetic device or structure manufactured to replace or enhance a biological structure. For example, in a total hip replacement procedure a prosthetic acetabular cup (implant) is used to replace or enhance a patients worn or damaged acetabulum. While the term “implant” is generally considered to denote a man-made structure (as contrasted with a transplant), for the purposes of this specification an implant can include a biological tissue or material transplanted to replace or enhance a biological structure.

For the purposes of this disclosure, the term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

For the purposes of this disclosure, the terms “distract,” “distracting,” or “distraction” are used to refer to displacement of a first point with respect to a second point. For example, the first point and the second point may correspond to surfaces of a joint. In some embodiments herein, a joint may be distracted, i.e., portions of the joint may be separated and/or moved with respect to one another to place the joint under tension. In some embodiments, a first portion of the joint be a surface of a scapula and a second portion of the joint may be a surface of a humerus such that separation occurs between the bones of the joint. In additional embodiments, a first portion of the joint may be a first portion of a humeral implant component or a humeral trial implant and a second portion of the joint may be a second portion of the humeral implant component or the humeral trial implant that is movable with respect to the first portion (e.g., a humeral component and a spacer). Accordingly, separation may occur between the portions of the humeral implant component or the humeral trial implant (i.e., intra-implant separation). Throughout the disclosure herein, the described embodiments may be collectively referred to as distraction of the joint.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly demarcated. For example, a reference to a surgeon also could apply, in some embodiments to a technician or nurse.

The systems, methods, and devices disclosed herein are particularly well adapted for surgical procedures that utilize surgical navigation systems, such as the CORI® surgical navigation system. CORI is a registered trademark of SMITH & NEPHEW, INC. of Memphis, TN.

### CASS Ecosystem Overview

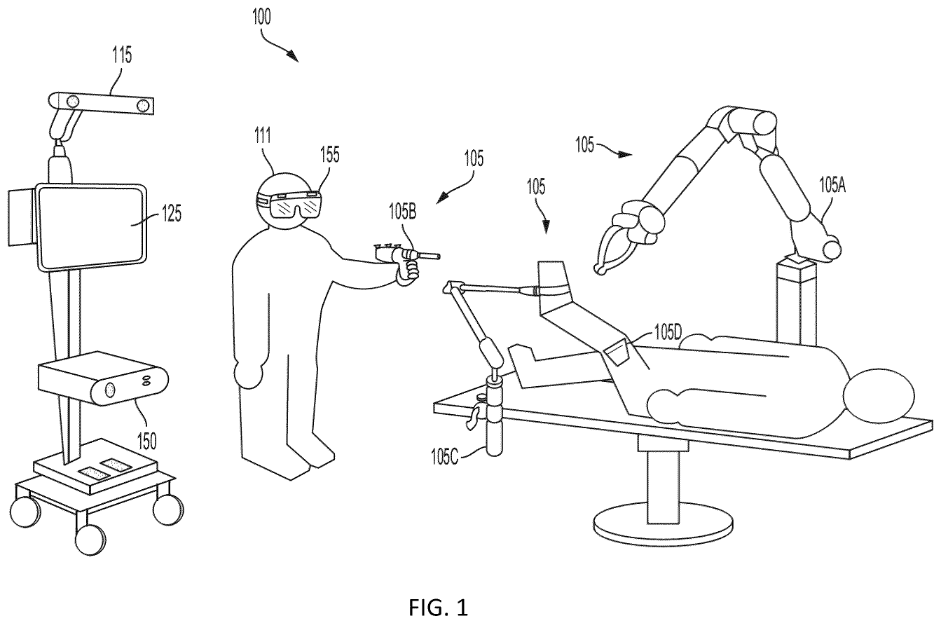

FIG. 1 provides an illustration of an example computer-assisted surgical system (CASS) 100, according to some embodiments. As described in further detail in the sections that follow, the CASS uses computers, robotics, and imaging technology to aid surgeons in performing orthopedic surgery procedures such as total knee arthroplasty (TKA), unicondylar knee arthroplasty (UKA), or total hip arthroplasty (THA). For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An Effector Platform 105 positions surgical tools relative to a patient during surgery. The exact components of the Effector Platform 105 will vary, depending on the embodiment employed. For example, for a knee surgery, the Effector Platform 105 may include an End Effector 105B that holds surgical tools or instruments during their use. The End Effector 105B may be a handheld device or instrument used by the surgeon (e.g., a CORI® hand piece or a cutting guide or jig) or, alternatively, the End Effector 105B can include a device or instrument held or positioned by a robotic arm 105A. While one robotic arm 105A is illustrated in FIG. 1, in some embodiments there may be multiple devices. As examples, there may be one robotic arm 105A on each side of an operating table T or two devices on one side of the table T. The robotic arm 105A may be mounted directly to the table T, be located next to the table T on a floor platform (not shown), mounted on a floor-to-ceiling pole, or mounted on a wall or ceiling of an operating room. The floor platform may be fixed or moveable. In one particular embodiment, the robotic arm 105A is mounted on a floor-to-ceiling pole located between the patient's legs or feet. In some embodiments, the End Effector 105B may include a suture holder or a stapler to assist in closing wounds. Further, in the case of two robotic arms 105A, the surgical computer 150 can drive the robotic arms 105A to work together to suture the wound at closure. Alternatively, the surgical computer 150 can drive one or more robotic arms 105A to staple the wound at closure.

The Effector Platform 105 can include a Limb Positioner 105C for positioning the patient's limbs during surgery. One example of a Limb Positioner 105C is the SMITH AND NEPHEW SPIDER2 system. The Limb Positioner 105C may be operated manually by the surgeon or alternatively change limb positions based on instructions received from the Surgical Computer 150 (described below). While one Limb Positioner 105C is illustrated in FIG. 1, in some embodiments there may be multiple devices. As examples, there may be one Limb Positioner 105C on each side of the operating table T or two devices on one side of the table T. The Limb Positioner 105C may be mounted directly to the table T, be located next to the table T on a floor platform (not shown), mounted on a pole, or mounted on a wall or ceiling of an operating room. In some embodiments, the Limb Positioner 105C can be used in non-conventional ways, such as a retractor or specific bone holder. The Limb Positioner 105C may include, as examples, an ankle boot, a soft tissue clamp, a bone clamp, or a soft-tissue retractor spoon, such as a hooked, curved, or angled blade. In some embodiments, the Limb Positioner 105C may include a suture holder to assist in closing wounds.

The Effector Platform 105 may include tools, such as a screwdriver, light or laser, to indicate an axis or plane, bubble level, pin driver, pin puller, plane checker, pointer, finger, or some combination thereof.

Resection Equipment 110 (not shown in FIG. 1) performs bone or tissue resection using, for example, mechanical, ultrasonic, or laser techniques. Examples of Resection Equipment 110 include drilling devices, burring devices, oscillatory sawing devices, vibratory impaction devices, reamers, ultrasonic bone cutting devices, radio frequency ablation devices, reciprocating devices (such as a rasp or broach), and laser ablation systems. In some embodiments, the Resection Equipment 110 is held and operated by the surgeon during surgery. In other embodiments, the Effector Platform 105 may be used to hold the Resection Equipment 110 during use.

The Effector Platform 105 also can include a cutting guide or jig 105D that is used to guide saws or drills used to resect tissue during surgery. Such cutting guides 105D can be formed integrally as part of the Effector Platform 105 or robotic arm 105A or cutting guides can be separate structures that can be matingly and/or removably attached to the Effector Platform 105 or robotic arm 105A. The Effector Platform 105 or robotic arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The Tracking System 115 uses one or more sensors to collect real-time position data that locates the patient's anatomy and surgical instruments. For example, for TKA procedures, the Tracking System may provide a location and orientation of the End Effector 105B during the procedure. In addition to positional data, data from the Tracking System 115 also can be used to infer velocity/acceleration of anatomy/instrumentation, which can be used for tool control. In some embodiments, the Tracking System 115 may use a tracker array attached to the End Effector 105B to determine the location and orientation of the End Effector 105B. The position of the End Effector 105B may be inferred based on the position and orientation of the Tracking System 115 and a known relationship in three-dimensional space between the Tracking System 115 and the End Effector 105B. Various types of tracking systems may be used in various embodiments of the present invention including, without limitation, Infrared (IR) tracking systems, electromagnetic (EM) tracking systems, video or image based tracking systems, and ultrasound registration and tracking systems. Using the data provided by the tracking system 115, the surgical computer 150 can detect objects and prevent collision. For example, the surgical computer 150 can prevent the robotic arm 105A and/or the End Effector 105B from colliding with soft tissue.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some embodiments, this can include stereoscopic, tri-scopic, quad-scopic, etc. imaging. In addition to the camera array, which in some embodiments is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system. In some embodiments, the camera may be mounted on the robotic arm 105A.

In some embodiments, specific objects can be manually registered by a surgeon with the system preoperatively or intraoperatively. For example, by interacting with a user interface, a surgeon may identify the starting location for a tool or a bone structure. By tracking fiducial marks associated with that tool or bone structure, or by using other conventional image tracking modalities, a processor may track that tool or bone as it moves through the environment in a three-dimensional model.

In some embodiments, certain markers, such as fiducial marks that identify individuals, important tools, or bones in the theater may include passive or active identifiers that can be picked up by a camera or camera array associated with the tracking system. For example, an IR LED can flash a pattern that conveys a unique identifier to the source of that pattern, providing a dynamic identification mark. Similarly, one-or two-dimensional optical codes (barcode, QR code, etc.) can be affixed to objects in the theater to provide passive identification that can occur based on image analysis. If these codes are placed asymmetrically on an object, they also can be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For example, in some embodiments, augmented reality (AR) headsets can be worn by surgeons and other staff to provide additional camera angles and tracking capabilities. In this case, the infrared/time of flight sensor data, which is predominantly used for hand/gesture detection, can build correspondence between the AR headset and the tracking system of the robotic system using sensor fusion techniques. This can be used to calculate a calibration matrix that relates the optical camera coordinate frame to the fixed holographic world frame.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient also can involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Bony landmarks can include, for example, the medial malleolus and lateral malleolus, the ends of the proximal femur and distal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process also can include determining various axes of a joint. For example, for a TKA the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

A Tissue Navigation System 120 (not shown in FIG. 1) provides the surgeon with intraoperative, real-time visualization for the patient's bone, cartilage, muscle, nervous, and/or vascular tissues surrounding the surgical area. Examples of systems that may be employed for tissue navigation include fluorescent imaging systems and ultrasound systems.

The Display 125 provides graphical user interfaces (GUIs) that display images collected by the Tissue Navigation System 120 as well other information relevant to the surgery. For example, in one embodiment, the Display 125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The Display 125 may include, for example, one or more computer monitors. As an alternative or supplement to the Display 125, one or more members of the surgical staff may wear an Augmented Reality (AR) Head Mounted Device (HMD). For example, in FIG. 1 the Surgeon 111 is wearing an AR HMD 155 that may, for example, overlay pre-operative image data on the patient or provide surgical planning suggestions. In one embodiment, a tracker array-mounted surgical tool could be detected by both the IR camera and an AR headset (HMD) using sensor fusion techniques without the need for any “intermediate” calibration rigs. This near-depth, time-of-flight sensing camera located in the HMD could be used for hand/gesture detection. The headset's sensor API can be used to expose IR and depth image data and carryout image processing using, for example, C++ with OpenCV. This approach allows the relationship between the CASS and the virtual coordinate frame to be determined and the headset sensor data (i.e., IR in combination with depth images) to isolate the CASS tracker arrays. The image processing system on the HMD can locate the surgical tool in a fixed holographic world frame and the CASS IR camera can locate the surgical tool relative to its camera coordinate frame. This relationship can be used to calculate a calibration matrix that relates the CASS IR camera coordinate frame to the fixed holographic world frame. This means that if a calibration matrix has previously been calculated, the surgical tool no longer needs to be visible to the AR headset. However, a recalculation may be necessary if the CASS camera is accidentally moved in the workflow. Various example uses of the AR HMD 155 in surgical procedures are detailed in the sections that follow.

Surgical Computer 150 provides control instructions to various components of the CASS 100, collects data from those components, and provides general processing for various data needed during surgery. In some embodiments, the Surgical Computer 150 is a general-purpose computer. In other embodiments, the Surgical Computer 150 may be a parallel computing platform that uses multiple central processing units (CPUs) or graphics processing units (GPU) to perform processing. In some embodiments, the Surgical Computer 150 is connected to a remote server over one or more computer networks (e.g., the Internet). The remote server can be used, for example, for storage of data or execution of computationally intensive processing tasks.

Various techniques generally known in the art can be used for connecting the Surgical Computer 150 to the other components of the CASS 100. Moreover, the computers can connect to the Surgical Computer 150 using a mix of technologies. For example, the End Effector 105B may connect to the Surgical Computer 150 over a wired (i.e., serial) connection. The Tracking System 115, Tissue Navigation System 120, and Display 125 can similarly be connected to the Surgical Computer 150 using wired connections. Alternatively, the Tracking System 115, Tissue Navigation System 120, and Display 125 may connect to the Surgical Computer 150 using wireless technologies such as, without limitation, Wi-Fi, Bluetooth, Near Field Communication (NFC), or ZigBee.

### Robotic Arm

In some embodiments, the CASS 100 includes a robotic arm 105A that serves as an interface to stabilize and hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The robotic arm 105A may have multiple degrees of freedom (like a Spider device) and have the ability to be locked in place (e.g., by a press of a button, voice activation, a surgeon removing a hand from the robotic arm, or other method).

In some embodiments, movement of the robotic arm 105A may be effectuated by use of a control panel built into the robotic arm system. For example, a display screen may include one or more input sources, such as physical buttons or a user interface having one or more icons, that direct movement of the robotic arm 105A. The surgeon or other healthcare professional may engage with the one or more input sources to position the robotic arm 105A when performing a surgical procedure.

A tool or an end effector 105B attached or integrated into a robotic arm 105A may include, without limitation, a burring device, a scalpel, a cutting device, a retractor, a joint tensioning device, or the like. In embodiments in which an end effector 105B is used, the end effector may be positioned at the end of the robotic arm 105A such that any motor control operations are performed within the robotic arm system. In embodiments in which a tool is used, the tool may be secured at a distal end of the robotic arm 105A, but motor control operation may reside within the tool itself.

The robotic arm 105A may be motorized internally to both stabilize the robotic arm, thereby preventing it from falling and hitting the patient, surgical table, surgical staff, etc., and to allow the surgeon to move the robotic arm without having to fully support its weight. While the surgeon is moving the robotic arm 105A, the robotic arm may provide some resistance to prevent the robotic arm from moving too fast or having too many degrees of freedom active at once. The position and the lock status of the robotic arm 105A may be tracked, for example, by a controller or the Surgical Computer 150.

In some embodiments, the robotic arm 105A can be moved by hand (e.g., by the surgeon) or with internal motors into its ideal position and orientation for the task being performed. In some embodiments, the robotic arm 105A may be enabled to operate in a “free” mode that allows the surgeon to position the arm into a desired position without being restricted. While in the free mode, the position and orientation of the robotic arm 105A may still be tracked as described above. In one embodiment, certain degrees of freedom can be selectively released upon input from user (e.g., surgeon) during specified portions of the surgical plan tracked by the Surgical Computer 150. Designs in which a robotic arm 105A is internally powered through hydraulics or motors or provides resistance to external manual motion through similar means can be described as powered robotic arms, while arms that are manually manipulated without power feedback, but which may be manually or automatically locked in place, may be described as passive robotic arms.

A robotic arm 105A or end effector 105B can include a trigger or other means to control the power of a saw or drill. Engagement of the trigger or other means by the surgeon can cause the robotic arm 105A or end effector 105B to transition from a motorized alignment mode to a mode where the saw or drill is engaged and powered on. Additionally, the CASS 100 can include a foot pedal (not shown) that causes the system to perform certain functions when activated. For example, the surgeon can activate the foot pedal to instruct the CASS 100 to place the robotic arm 105A or end effector 105B in an automatic mode that brings the robotic arm or end effector into the proper position with respect to the patient's anatomy in order to perform the necessary resections. The CASS 100 also can place the robotic arm 105A or end effector 105B in a collaborative mode that allows the surgeon to manually manipulate and position the robotic arm or end effector into a particular location. The collaborative mode can be configured to allow the surgeon to move the robotic arm 105A or end effector 105B medially or laterally, while restricting movement in other directions. As discussed, the robotic arm 105A or end effector 105B can include a cutting device (saw, drill, and burr) or a cutting guide or jig 105D that will guide a cutting device. In other embodiments, movement of the robotic arm 105A or robotically controlled end effector 105B can be controlled entirely by the CASS 100 without any, or with only minimal, assistance or input from a surgeon or other medical professional. In still other embodiments, the movement of the robotic arm 105A or robotically controlled end effector 105B can be controlled remotely by a surgeon or other medical professional using a control mechanism separate from the robotic arm or robotically controlled end effector device, for example using a joystick or interactive monitor or display control device.

A robotic arm 105A may be used for holding the retractor. For example, in one embodiment, the robotic arm 105A may be moved into the desired position by the surgeon. At that point, the robotic arm 105A may lock into place. In some embodiments, the robotic arm 105A is provided with data regarding the patient's position, such that if the patient moves, the robotic arm can adjust the retractor position accordingly. In some embodiments, multiple robotic arms may be used, thereby allowing multiple retractors to be held or for more than one activity to be performed simultaneously (e.g., retractor holding & reaming).

The robotic arm 105A may also be used to help stabilize the surgeon's hand while making a femoral neck cut. In this application, control of the robotic arm 105A may impose certain restrictions to prevent soft tissue damage from occurring. For example, in one embodiment, the Surgical Computer 150 tracks the position of the robotic arm 105A as it operates. If the tracked location approaches an area where tissue damage is predicted, a command may be sent to the robotic arm 105A causing it to stop. Alternatively, where the robotic arm 105A is automatically controlled by the Surgical Computer 150, the Surgical Computer may ensure that the robotic arm is not provided with any instructions that cause it to enter areas where soft tissue damage is likely to occur. The Surgical Computer 150 may impose certain restrictions on the surgeon to prevent the surgeon from reaming too far into the medial wall of the acetabulum or reaming at an incorrect angle or orientation.

In some embodiments, the robotic arm 105A may be used to hold a cup impactor at a desired angle or orientation during cup impaction. When the final position has been achieved, the robotic arm 105A may prevent any further seating to prevent damage to the pelvis.

The surgeon may use the robotic arm 105A to position the broach handle at the desired position and allow the surgeon to impact the broach into the femoral canal at the desired orientation. In some embodiments, once the Surgical Computer 150 receives feedback that the broach is fully seated, the robotic arm 105A may restrict the handle to prevent further advancement of the broach.

The robotic arm 105A may also be used for resurfacing applications. For example, the robotic arm 105A may stabilize the surgeon while using traditional instrumentation and provide certain restrictions or limitations to allow for proper placement of implant components (e.g., guide wire placement, chamfer cutter, sleeve cutter, plan cutter, etc.). Where only a burr is employed, the robotic arm 105A may stabilize the surgeon's handpiece and may impose restrictions on the handpiece to prevent the surgeon from removing unintended bone in contravention of the surgical plan.

The robotic arm 105A may be a passive arm. As an example, the robotic arm 105A may be a CIRQ robot arm available from Brainlab AG. CIRQ is a registered trademark of Brainlab AG, Olof-Palme-Str. 9 81829, München, FED REP of GERMANY. In one particular embodiment, the robotic arm 105A is an intelligent holding arm as disclosed in U.S. patent application Ser. No. 15/525,585 to Krinninger et al., U.S. patent application Ser. No. 15/561,042 to Nowatschin et al., U.S. patent application Ser. No. 15/561,048 to Nowatschin et al., and U.S. Pat. No. 10,342,636 to Nowatschin et al., the entire contents of each of which is herein incorporated by reference.

### Surgical Procedure Data Generation and Collection

The various services that are provided by medical professionals to treat a clinical condition are collectively referred to as an “episode of care.” For a particular surgical intervention, the episode of care can include three phases: pre-operative, intra-operative, and post-operative. During each phase, data is collected or generated that can be used to analyze the episode of care in order to understand various features of the procedure and identify patterns that may be used, for example, in training models to make decisions with minimal human intervention. The data collected over the episode of care may be stored at the Surgical Computer 150 or the Surgical Data Server 180 as a complete dataset. Thus, for each episode of care, a dataset exists that comprises all of the data collectively pre-operatively about the patient, all of the data collected or stored by the CASS 100 intra-operatively, and any post-operative data provided by the patient or by a healthcare professional monitoring the patient.

As explained in further detail, the data collected during the episode of care may be used to enhance performance of the surgical procedure or to provide a holistic understanding of the surgical procedure and the patient outcomes. For example, in some embodiments, the data collected over the episode of care may be used to generate a surgical plan. In one embodiment, a high-level, pre-operative plan is refined intra-operatively as data is collected during surgery. In this way, the surgical plan can be viewed as dynamically changing in real-time or near real-time as new data is collected by the components of the CASS 100. In other embodiments, pre-operative images or other input data may be used to develop a robust plan preoperatively that is simply executed during surgery. In this case, the data collected by the CASS 100 during surgery may be used to make recommendations that ensure that the surgeon stays within the pre-operative surgical plan. For example, if the surgeon is unsure how to achieve a certain prescribed cut or implant alignment, the Surgical Computer 150 can be queried for a recommendation. In still other embodiments, the pre-operative and intra-operative planning approaches can be combined such that a robust pre-operative plan can be dynamically modified, as necessary or desired, during the surgical procedure. In some embodiments, a biomechanics-based model of patient anatomy contributes simulation data to be considered by the CASS 100 in developing preoperative, intraoperative, and post-operative/rehabilitation procedures to optimize implant performance outcomes for the patient.

Aside from changing the surgical procedure itself, the data gathered during the episode of care may be used as an input to other procedures ancillary to the surgery. For example, in some embodiments, implants can be designed using episode of care data. Example data-driven techniques for designing, sizing, and fitting implants are described in U.S. Pat. No. 10,064,686, filed Aug. 15, 2011, and entitled “Systems and Methods for Optimizing Parameters for Orthopaedic Procedures”; U.S. Pat. No. 10,102,309, filed Jul. 20, 2012 and entitled “Systems and Methods for Optimizing Fit of an Implant to Anatomy”; and U.S. Pat. No. 8,078,440, filed Sep. 19, 2008 and entitled “Operatively Tuning Implants for Increased Performance,” the entire contents of each of which are hereby incorporated by reference into this patent application.

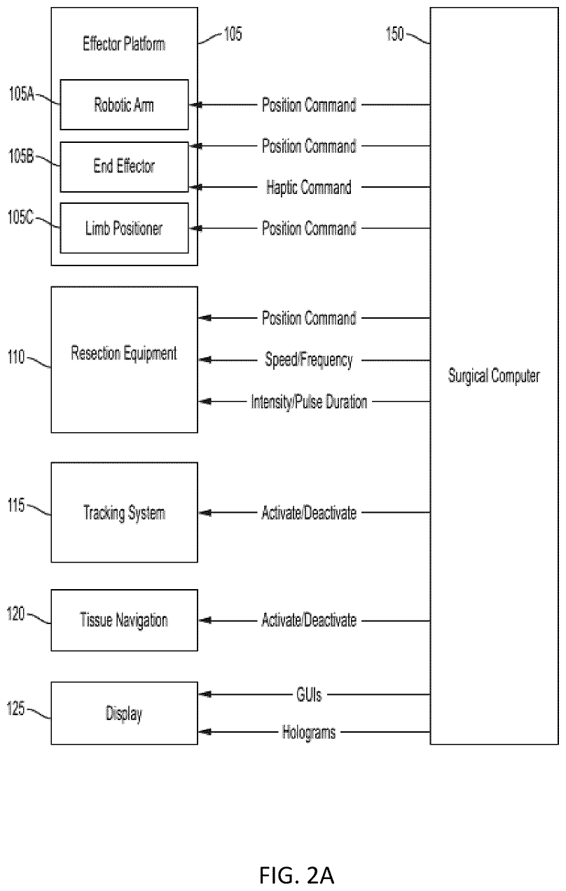

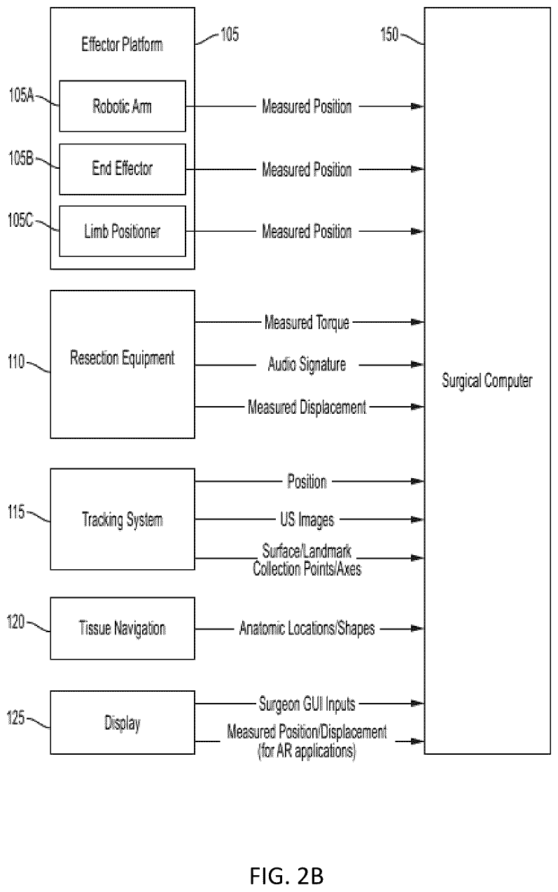

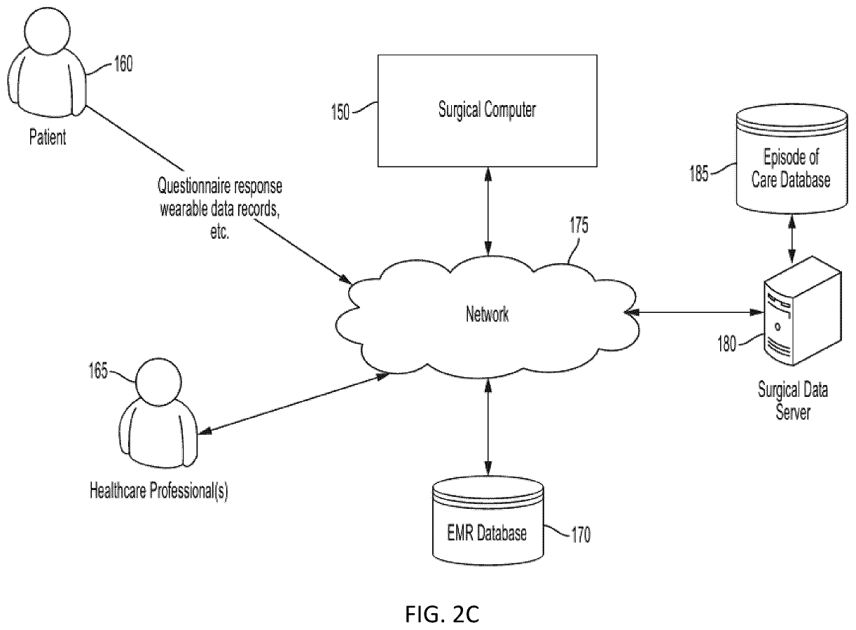

Furthermore, the data can be used for educational, training, or research purposes. For example, using the network-based approach described below in FIG. 2C, other doctors or students can remotely view surgeries in interfaces that allow them to selectively view data as it is collected from the various components of the CASS 100. After the surgical procedure, similar interfaces may be used to “playback” a surgery for training or other educational purposes, or to identify the source of any issues or complications with the procedure.

Data acquired during the pre-operative phase generally includes all information collected or generated prior to the surgery. Thus, for example, information about the patient may be acquired from a patient intake form or electronic medical record (EMR). Examples of patient information that may be collected include, without limitation, patient demographics, diagnoses, medical histories, progress notes, vital signs, medical history information, allergies, and lab results. The pre-operative data may also include images related to the anatomical area of interest. These images may be captured, for example, using Magnetic Resonance Imaging (MRI), Computed Tomography (CT), X-ray, ultrasound, or any other modality known in the art. The pre-operative data may also comprise quality of life data captured from the patient. For example, in one embodiment, pre-surgery patients use a mobile application (“app”) to answer questionnaires regarding their current quality of life. In some embodiments, preoperative data used by the CASS 100 includes demographic, anthropometric, cultural, or other specific traits about a patient that can coincide with activity levels and specific patient activities to customize the surgical plan to the patient. For example, certain cultures or demographics may be more likely to use a toilet that requires squatting on a daily basis.

FIGS. 2A and 2B provide examples of data that may be acquired during the intra-operative phase of an episode of care. These examples are based on the various components of the CASS 100 described above with reference to FIG. 1; however, it should be understood that other types of data may be used based on the types of equipment used during surgery and their use.

FIG. 2A shows examples of some of the control instructions that the Surgical Computer 150 provides to other components of the CASS 100, according to some embodiments. Note that the example of FIG. 2A assumes that the components of the Effector Platform 105 are each controlled directly by the Surgical Computer 150. In embodiments where a component is manually controlled by the Surgeon 111, instructions may be provided on the Display 125 or AR HMD 155 instructing the Surgeon 111 how to move the component.

The various components included in the Effector Platform 105 are controlled by the Surgical Computer 150 providing position commands that instruct the component where to move within a coordinate system. In some embodiments, the Surgical Computer 150 provides the Effector Platform 105 with instructions defining how to react when a component of the Effector Platform 105 deviates from a surgical plan. These commands are referenced in FIG. 2A as “haptic” commands. For example, the End Effector 105B may provide a force to resist movement outside of an area where resection is planned. Other commands that may be used by the Effector Platform 105 include vibration and audio cues.

In some embodiments, the end effectors 105B of the robotic arm 105A are operatively coupled with cutting guide 105D. In response to an anatomical model of the surgical scene, the robotic arm 105A can move the end effectors 105B and the cutting guide 105D into position to match the location of the femoral or tibial cut to be performed in accordance with the surgical plan. This can reduce the likelihood of error, allowing the vision system and a processor utilizing that vision system to implement the surgical plan to place a cutting guide 105D at the precise location and orientation relative to the tibia or femur to align a cutting slot of the cutting guide with the cut to be performed according to the surgical plan. Then, a surgeon can use any suitable tool, such as an oscillating or rotating saw or drill to perform the cut (or drill a hole) with perfect placement and orientation because the tool is mechanically limited by the features of the cutting guide 105D. In some embodiments, the cutting guide 105D may include one or more pin holes that are used by a surgeon to drill and screw or pin the cutting guide into place before performing a resection of the patient tissue using the cutting guide. This can free the robotic arm 105A or ensure that the cutting guide 105D is fully affixed without moving relative to the bone to be resected. For example, this procedure can be used to make the first distal cut of the femur during a total knee arthroplasty. In some embodiments, where the arthroplasty is a hip arthroplasty, cutting guide 105D can be fixed to the femoral head or the acetabulum for the respective hip arthroplasty resection. It should be understood that any arthroplasty that utilizes precise cuts can use the robotic arm 105A and/or cutting guide 105D in this manner.

The Resection Equipment 110 is provided with a variety of commands to perform bone or tissue operations. As with the Effector Platform 105, position information may be provided to the Resection Equipment 110 to specify where it should be located when performing resection. Other commands provided to the Resection Equipment 110 may be dependent on the type of resection equipment. For example, for a mechanical or ultrasonic resection tool, the commands may specify the speed and frequency of the tool. For Radiofrequency Ablation (RFA) and other laser ablation tools, the commands may specify intensity and pulse duration.

Some components of the CASS 100 do not need to be directly controlled by the Surgical Computer 150; rather, the Surgical Computer 150 only needs to activate the component, which then executes software locally specifying the manner in which to collect data and provide it to the Surgical Computer 150. In the example of FIG. 2A, there are two components that are operated in this manner: the Tracking System 115 and the Tissue Navigation System 120.

The Surgical Computer 150 provides the Display 125 with any visualization that is needed by the Surgeon 111 during surgery. For monitors, the Surgical Computer 150 may provide instructions for displaying images, GUIs, etc. using techniques known in the art. The display 125 can include various portions of the workflow of a surgical plan. During the registration process, for example, the display 125 can show a preoperatively constructed 3D bone model and depict the locations of the probe as the surgeon uses the probe to collect locations of anatomical landmarks on the patient. The display 125 can include information about the surgical target area. For example, in connection with a TKA, the display 125 can depict the mechanical and anatomical axes of the femur and tibia. The display 125 can depict varus and valgus angles for the knee joint based on a surgical plan, and the CASS 100 can depict how such angles will be affected if contemplated revisions to the surgical plan are made. Accordingly, the display 125 is an interactive interface that can dynamically update and display how changes to the surgical plan would impact the procedure and the final position and orientation of implants installed on bone.

As the workflow progresses to preparation of bone cuts or resections, the display 125 can depict the planned or recommended bone cuts before any cuts are performed. The surgeon 111 can manipulate the image display to provide different anatomical perspectives of the target area and can have the option to alter or revise the planned bone cuts based on intraoperative evaluation of the patient. The display 125 can depict how the chosen implants would be installed on the bone if the planned bone cuts are performed. If the surgeon 111 choses to change the previously planned bone cuts, the display 125 can depict how the revised bone cuts would change the position and orientation of the implant when installed on the bone.

The display 125 can provide the surgeon 111 with a variety of data and information about the patient, the planned surgical intervention, and the implants. Various patient-specific information can be displayed, including real-time data concerning the patient's health such as heart rate, blood pressure, etc. The display 125 also can include information about the anatomy of the surgical target region including the location of landmarks, the current state of the anatomy (e.g., whether any resections have been made, the depth and angles of planned and executed bone cuts), and future states of the anatomy as the surgical plan progresses. The display 125 also can provide or depict additional information about the surgical target region. For a TKA, the display 125 can provide information about the gaps (e.g., gap balancing) between the femur and tibia and how such gaps will change if the planned surgical plan is carried out. For a TKA, the display 125 can provide additional relevant information about the knee joint such as data about the joint's tension (e.g., ligament laxity) and information concerning rotation and alignment of the joint. The display 125 can depict how the planned implants' locations and positions will affect the patient as the knee joint is flexed. The display 125 can depict how the use of different implants or the use of different sizes of the same implant will affect the surgical plan and preview how such implants will be positioned on the bone. The CASS 100 can provide such information for each of the planned bone resections in a TKA or THA. In a TKA, the CASS 100 can provide robotic control for one or more of the planned bone resections. For example, the CASS 100 can provide robotic control only for the initial distal femur cut, and the surgeon 111 can manually perform other resections (anterior, posterior and chamfer cuts) using conventional means, such as a 4-in-1 cutting guide or jig 105D.

The display 125 can employ different colors to inform the surgeon of the status of the surgical plan. For example, un-resected bone can be displayed in a first color, resected bone can be displayed in a second color, and planned resections can be displayed in a third color. Implants can be superimposed onto the bone in the display 125, and implant colors can change or correspond to different types or sizes of implants.

The information and options depicted on the display 125 can vary depending on the type of surgical procedure being performed. Further, the surgeon 111 can request or select a particular surgical workflow display that matches or is consistent with his or her surgical plan preferences. For example, for a surgeon 111 who typically performs the tibial cuts before the femoral cuts in a TKA, the display 125 and associated workflow can be adapted to take this preference into account. The surgeon 111 also can preselect that certain steps be included or deleted from the standard surgical workflow display. For example, if a surgeon 111 uses resection measurements to finalize an implant plan but does not analyze ligament gap balancing when finalizing the implant plan, the surgical workflow display can be organized into modules, and the surgeon can select which modules to display and the order in which the modules are provided based on the surgeon's preferences or the circumstances of a particular surgery. Modules directed to ligament and gap balancing, for example, can include pre- and post-resection ligament/gap balancing, and the surgeon 111 can select which modules to include in their default surgical plan workflow depending on whether they perform such ligament and gap balancing before or after (or both) bone resections are performed.

For more specialized display equipment, such as AR HMDs, the Surgical Computer 150 may provide images, text, etc. using the data format supported by the equipment. For example, if the Display 125 is a holography device such as the Microsoft HoloLens™ or Magic Leap One™, the Surgical Computer 150 may use the HoloLens Application Program Interface (API) to send commands specifying the position and content of holograms displayed in the field of view of the Surgeon 111.

In some embodiments, one or more surgical planning models may be incorporated into the CASS 100 and used in the development of the surgical plans provided to the surgeon 111. The term “surgical planning model” refers to software that simulates the biomechanics performance of anatomy under various scenarios to determine the optimal way to perform cutting and other surgical activities. For example, for knee replacement surgeries, the surgical planning model can measure parameters for functional activities, such as deep knee bends, gait, etc., and select cut locations on the knee to optimize implant placement. One example of a surgical planning model is the LIFEMOD™ simulation software from SMITH AND NEPHEW, INC. In some embodiments, the Surgical Computer 150 includes computing architecture that allows full execution of the surgical planning model during surgery (e.g., a GPU-based parallel processing environment). In other embodiments, the Surgical Computer 150 may be connected over a network to a remote computer that allows such execution, such as a Surgical Data Server 180 (see FIG. 2C). As an alternative to full execution of the surgical planning model, in some embodiments, a set of transfer functions are derived that simplify the mathematical operations captured by the model into one or more predictor equations. Then, rather than execute the full simulation during surgery, the predictor equations are used. Further details on the use of transfer functions are described in WIPO Publication No. 2020/037308, filed Aug. 19, 2019, entitled “Patient Specific Surgical Method and System,” the entirety of which is incorporated herein by reference.

FIG. 2B shows examples of some of the types of data that can be provided to the Surgical Computer 150 from the various components of the CASS 100. In some embodiments, the components may stream data to the Surgical Computer 150 in real-time or near real-time during surgery. In other embodiments, the components may queue data and send it to the Surgical Computer 150 at set intervals (e.g., every second). Data may be communicated using any format known in the art. Thus, in some embodiments, the components all transmit data to the Surgical Computer 150 in a common format. In other embodiments, each component may use a different data format, and the Surgical Computer 150 is configured with one or more software applications that enable translation of the data. Thus, data may be acquired by the Surgical Computer 150 using a variety of systems, formats, and modalities.

In general, the Surgical Computer 150 may serve as the central point where CASS data is collected. The exact content of the data will vary depending on the source. For example, each component of the Effector Platform 105 provides a measured position to the Surgical Computer 150. Thus, by comparing the measured position to a position originally specified by the Surgical Computer 150 (see FIG. 2B), the Surgical Computer can identify deviations that take place during surgery.

The Resection Equipment 110 can send various types of data to the Surgical Computer 150 depending on the type of equipment used. Example data types that may be sent include the measured torque, audio signatures, and measured displacement values. Similarly, the Tracking Technology 115 can provide different types of data depending on the tracking methodology employed. Example tracking data types include position values for tracked items (e.g., anatomy, tools, etc.), ultrasound images, and surface or landmark collection points or axes. The Tissue Navigation System 120 provides the Surgical Computer 150 with anatomic locations, shapes, etc. as the system operates.

Although the Display 125 generally is used for outputting data for presentation to the user, it may also provide data to the Surgical Computer 150. For example, for embodiments where a monitor is used as part of the Display 125, the Surgeon 111 may interact with a GUI to provide inputs which are sent to the Surgical Computer 150 for further processing. For AR applications, the measured position and displacement of the HMD may be sent to the Surgical Computer 150 so that it can update the presented view as needed.

During the post-operative phase of the episode of care, various types of data can be collected to quantify the overall improvement or deterioration in the patient's condition as a result of the surgery. The data can take the form of, for example, self-reported information reported by patients via questionnaires. For example, in the context of a knee replacement surgery, functional status can be measured with an Oxford Knee Score questionnaire, and the post-operative quality of life can be measured with a EQ5D-5L questionnaire. Other examples in the context of a hip replacement surgery may include the Oxford Hip Score, Harris Hip Score, and WOMAC (Western Ontario and McMaster Universities Osteoarthritis index). Such questionnaires can be administered, for example, by a healthcare professional directly in a clinical setting or using a mobile app that allows the patient to respond to questions directly. In some embodiments, the patient may be outfitted with one or more wearable devices that collect data relevant to the surgery. For example, following a knee surgery, the patient may be outfitted with a knee brace that includes sensors that monitor knee positioning, flexibility, etc. This information can be collected and transferred to the patient's mobile device for review by the surgeon to evaluate the outcome of the surgery and address any issues. In some embodiments, one or more cameras can capture and record the motion of a patient's body segments during specified activities postoperatively. This motion capture can be compared to a biomechanics model to better understand the functionality of the patient's joints and better predict progress in recovery and identify any possible revisions that may be needed.

The post-operative stage of the episode of care can continue over the entire life of a patient. For example, in some embodiments, the Surgical Computer 150 or other components comprising the CASS 100 can continue to receive and collect data relevant to a surgical procedure after the procedure has been performed. This data may include, for example, images, answers to questions, “normal” patient data (e.g., blood type, blood pressure, conditions, medications, etc.), biometric data (e.g., gait, etc.), and objective and subjective data about specific issues (e.g., knee or hip joint pain). This data may be explicitly provided to the Surgical Computer 150 or other CASS component by the patient or the patient's physician(s). Alternatively, or additionally, the Surgical Computer 150 or other CASS component can monitor the patient's EMR and retrieve relevant information as it becomes available. This longitudinal view of the patient's recovery allows the Surgical Computer 150 or other CASS component to provide a more objective analysis of the patient's outcome to measure and track success or lack of success for a given procedure. For example, a condition experienced by a patient long after the surgical procedure can be linked back to the surgery through a regression analysis of various data items collected during the episode of care. This analysis can be further enhanced by performing the analysis on groups of patients that had similar procedures and/or have similar anatomies.

In some embodiments, data is collected at a central location to provide for easier analysis and use. Data can be manually collected from various CASS components in some instances. For example, a portable storage device (e.g., USB stick) can be attached to the Surgical Computer 150 into order to retrieve data collected during surgery. The data can then be transferred, for example, via a desktop computer to the centralized storage. Alternatively, in some embodiments, the Surgical Computer 150 is connected directly to the centralized storage via a Network 175 as shown in FIG. 2C.

FIG. 2C illustrates a “cloud-based” implementation in which the Surgical Computer 150 is connected to a Surgical Data Server 180 via a Network 175. This Network 175 may be, for example, a private intranet or the Internet. In addition to the data from the Surgical Computer 150, other sources can transfer relevant data to the Surgical Data Server 180. The example of FIG. 2C shows three additional data sources: the Patient 160, Healthcare Professional(s) 165, and an EMR Database 170. Thus, the Patient 160 can send pre-operative and post-operative data to the Surgical Data Server 180, for example, using a mobile app. The Healthcare Professional(s) 165 includes the surgeon and his or her staff as well as any other professionals working with Patient 160 (e.g., a personal physician, a rehabilitation specialist, etc.). It should also be noted that the EMR Database 170 may be used for both pre-operative and post-operative data. For example, assuming that the Patient 160 has given adequate permissions, the Surgical Data Server 180 may collect the EMR of the Patient pre-surgery. Then, the Surgical Data Server 180 may continue to monitor the EMR for any updates post-surgery.

At the Surgical Data Server 180, an Episode of Care Database 185 is used to store the various data collected over a patient's episode of care. The Episode of Care Database 185 may be implemented using any technique known in the art. For example, in some embodiments, a SQL-based database may be used where all of the various data items are structured in a manner that allows them to be readily incorporated in two SQL's collection of rows and columns. However, in other embodiments a No-SQL database may be employed to allow for unstructured data, while providing the ability to rapidly process and respond to queries. As is understood in the art, the term “No-SQL” is used to define a class of data stores that are non-relational in their design. Various types of No-SQL databases may generally be grouped according to their underlying data model. These groupings may include databases that use column-based data models (e.g., Cassandra), document-based data models (e.g., MongoDB), key-value based data models (e.g., Redis), and/or graph-based data models (e.g., Allego). Any type of No-SQL database may be used to implement the various embodiments described herein and, in some embodiments, the different types of databases may support the Episode of Care Database 185.

Data can be transferred between the various data sources and the Surgical Data Server 180 using any data format and transfer technique known in the art. It should be noted that the architecture shown in FIG. 2C allows transmission from the data source to the Surgical Data Server 180, as well as retrieval of data from the Surgical Data Server 180 by the data sources. For example, as explained in detail below, in some embodiments, the Surgical Computer 150 may use data from past surgeries, machine learning models, etc. to help guide the surgical procedure.

In some embodiments, the Surgical Computer 150 or the Surgical Data Server 180 may execute a de-identification process to ensure that data stored in the Episode of Care Database 185 meets Health Insurance Portability and Accountability Act (HIPAA) standards or other requirements mandated by law. HIPAA provides a list of certain identifiers that must be removed from data during de-identification. The aforementioned de-identification process can scan for these identifiers in data that is transferred to the Episode of Care Database 185 for storage. For example, in one embodiment, the Surgical Computer 150 executes the de-identification process just prior to initiating transfer of a particular data item or set of data items to the Surgical Data Server 180. In some embodiments, a unique identifier is assigned to data from a particular episode of care to allow for re-identification of the data if necessary.

Although FIGS. 2A-C discuss data collection in the context of a single episode of care, it should be understood that the general concept can be extended to data collection from multiple episodes of care. For example, surgical data may be collected over an entire episode of care each time a surgery is performed with the CASS 100 and stored at the Surgical Computer 150 or at the Surgical Data Server 180. As explained in further detail below, a robust database of episode of care data allows the generation of optimized values, measurements, distances, or other parameters and other recommendations related to the surgical procedure. In some embodiments, the various datasets are indexed in the database or other storage medium in a manner that allows for rapid retrieval of relevant information during the surgical procedure. For example, in one embodiment, a patient-centric set of indices may be used so that data pertaining to a particular patient or a set of patients similar to a particular patient can be readily extracted. This concept can be similarly applied to surgeons, implant characteristics, CASS component versions, etc.

Further details of the management of episode of care data are described in U.S. Pat. No. 11,532,402, filed Apr. 13, 2020, and entitled “METHODS AND SYSTEMS FOR PROVIDING AN EPISODE OF CARE,” the entirety of which is incorporated herein by reference.

### Using the Point Probe to Acquire High-Resolution of Key Areas During Hip Surgeries

Use of the point probe is described in U.S. patent application Ser. No. 14/455,742 entitled “Systems and Methods for Planning and Performing Image Free Implant Revision Surgery,” the entirety of which is incorporated herein by reference. Briefly, an optically tracked point probe may be used to map the actual surface of the target bone that needs a new implant. Mapping is performed after removal of the defective or worn-out implant, as well as after removal of any diseased or otherwise unwanted bone. A plurality of points is collected on the bone surfaces by brushing or scraping the entirety of the remaining bone with the tip of the point probe. This is referred to as tracing or “painting” the bone. The collected points are used to create a three-dimensional model or surface map of the bone surfaces in the computerized planning system. The created 3D model of the remaining bone is then used as the basis for planning the procedure and necessary implant sizes. An alternative technique that uses X-rays to determine a 3D model is described in U.S. patent application Ser. No. 16/387,151, filed Apr. 17, 2019 and entitled “Three-Dimensional Selective Bone Matching” and U.S. patent application Ser. No. 16/789,430, filed Feb. 13, 2020 and entitled “Three-Dimensional Selective Bone Matching,” the entirety of each of which is incorporated herein by reference.

For hip applications, the point probe painting can be used to acquire high resolution data in key areas such as the acetabular rim and acetabular fossa. This can allow a surgeon to obtain a detailed view before beginning to ream. For example, in one embodiment, the point probe may be used to identify the floor (fossa) of the acetabulum. As is well understood in the art, in hip surgeries, it is important to ensure that the floor of the acetabulum is not compromised during reaming so as to avoid destruction of the medial wall. If the medial wall were inadvertently destroyed, the surgery would require the additional step of bone grafting. With this in mind, the information from the point probe can be used to provide operating guidelines to the acetabular reamer during surgical procedures. For example, the acetabular reamer may be configured to provide haptic feedback to the surgeon when he or she reaches the floor or otherwise deviates from the surgical plan. Alternatively, the CASS 100 may automatically stop the reamer when the floor is reached or when the reamer is within a threshold distance.

As an additional safeguard, the thickness of the area between the acetabulum and the medial wall could be estimated. For example, once the acetabular rim and acetabular fossa has been painted and registered to the pre-operative 3D model, the thickness can readily be estimated by comparing the location of the surface of the acetabulum to the location of the medial wall. Using this knowledge, the CASS 100 may provide alerts or other responses in the event that any surgical activity is predicted to protrude through the acetabular wall while reaming.

The point probe may also be used to collect high resolution data of common reference points used in orienting the 3D model to the patient. For example, for pelvic plane landmarks like the ASIS and the pubic symphysis, the surgeon may use the point probe to paint the bone to represent a true pelvic plane. Given a more complete view of these landmarks, the registration software has more information to orient the 3D model.

The point probe may also be used to collect high-resolution data describing the proximal femoral reference point that could be used to increase the accuracy of implant placement. For example, the relationship between the tip of the Greater Trochanter (GT) and the center of the femoral head is commonly used as reference point to align the femoral component during hip arthroplasty. The alignment is highly dependent on proper location of the GT; thus, in some embodiments, the point probe is used to paint the GT to provide a high-resolution view of the area. Similarly, in some embodiments, it may be useful to have a high-resolution view of the Lesser Trochanter (LT). For example, during hip arthroplasty, the Dorr Classification helps to select a stem that will maximize the ability of achieving a press-fit during surgery to prevent micromotion of femoral components post-surgery and ensure optimal bony ingrowth. As is generated understood in the art, the Dorr Classification measures the ratio between the canal width at the LT and the canal width 10 cm below the LT. The accuracy of the classification is highly dependent on the correct location of the relevant anatomy. Thus, it may be advantageous to paint the LT to provide a high-resolution view of the area.

In some embodiments, the point probe is used to paint the femoral neck to provide high-resolution data that allows the surgeon to better understand where to make the neck cut. The navigation system can then guide the surgeon as they perform the neck cut. For example, as understood in the art, the femoral neck angle is measured by placing one line down the center of the femoral shaft and a second line down the center of the femoral neck. Thus, a high-resolution view of the femoral neck (and possibly the femoral shaft as well) would provide a more accurate calculation of the femoral neck angle.

High-resolution femoral head neck data also could be used for a navigated resurfacing procedure where the software/hardware aids the surgeon in preparing the proximal femur and placing the femoral component. As is generally understood in the art, during hip resurfacing, the femoral head and neck are not removed; rather, the head is trimmed and capped with a smooth metal covering. In this case, it would be advantageous for the surgeon to paint the femoral head and cap so that an accurate assessment of their respective geometries can be understood and used to guide trimming and placement of the femoral component.

### Registration of Pre-Operative Data to Patient Anatomy Using the Point Probe

As noted above, in some embodiments, a 3D model is developed during the pre-operative stage based on 2D or 3D images of the anatomical area of interest. In such embodiments, registration between the 3D model and the surgical site is performed prior to the surgical procedure. The registered 3D model may be used to track and measure the patient's anatomy and surgical tools intraoperatively.

During the surgical procedure, landmarks are acquired to facilitate registration of this pre-operative 3D model to the patient's anatomy. For knee procedures, these points could comprise the femoral head center, distal femoral axis point, medial and lateral epicondyles, medial and lateral malleolus, proximal tibial mechanical axis point, and tibial A/P direction. For hip procedures these points could comprise the anterior superior iliac spine (ASIS), the pubic symphysis, points along the acetabular rim and within the hemisphere, the greater trochanter (GT), and the lesser trochanter (LT).

In a revision surgery, the surgeon may paint certain areas that contain anatomical defects to allow for better visualization and navigation of implant insertion. These defects can be identified based on analysis of the pre-operative images. For example, in one embodiment, each pre-operative image is compared to a library of images showing “healthy” anatomy (i.e., without defects). Any significant deviations between the patient's images and the healthy images can be flagged as a potential defect. Then, during surgery, the surgeon can be warned of the possible defect via a visual alert on the display 125 of the CASS 100. The surgeon can then paint the area to provide further detail regarding the potential defect to the Surgical Computer 150.

In some embodiments, the surgeon may use a non-contact method for registration of bony anatomy intra-incision. For example, in one embodiment, laser scanning is employed for registration. A laser stripe is projected over the anatomical area of interest and the height variations of the area are detected as changes in the line. Other non-contact optical methods, such as white light interferometry or ultrasound, may alternatively be used for surface height measurement or to register the anatomy. For example, ultrasound technology may be beneficial where there is soft tissue between the registration point and the bone being registered (e.g., ASIS, pubic symphysis in hip surgeries), thereby providing for a more accurate definition of anatomic planes.

### Femur Tracking During Navigated Total Hip Arthroplasty

When a Total Hip Arthroplasty (THA) procedure is performed under the assistance of navigation, the pelvis and femur may be tracked in six degrees of freedom (e.g., x, y, z, roll, pitch, and yaw) using tracking markers (e.g., tracking arrays) affixed to the anatomy. In general, attachment of optical tracking arrays may require reliable line-of-site from the attachment location to the camera and stable attachment to the tracked anatomy. Furthermore, the tracking array may be configured to not obstruct the surgeon and/or any required tools (e.g., a robotic arm) during the procedure. Tracking arrays may be attached to the pelvis along the Iliac Crest, which is accessible and not overly obstructive during standard THA surgical steps. Accessibility for affixing a femoral array may be more challenging given the limited access and visibility, especially when using a minimally invasive Direct Anterior (DA) approach.

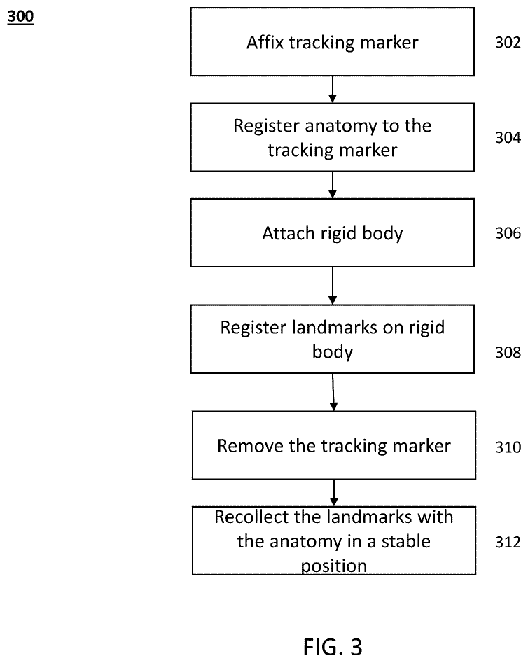

FIG. 3 depicts a flow chart for a method 300 of obtaining six degrees of freedom measurement data after a stable tracking marker has been removed from the anatomy in accordance with an embodiment.

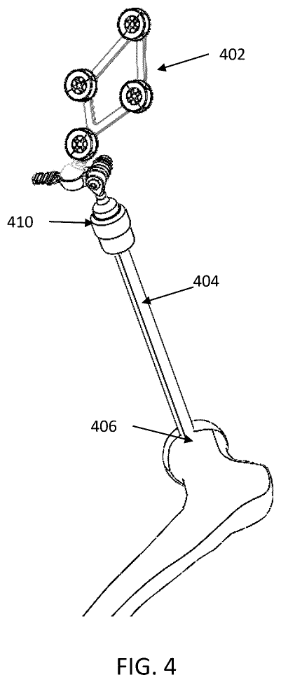

The method 300 may include affixing 302 a stable tracking marker (e.g., a tracking array) to the anatomy. As illustrated in FIG. 4, the tracking marker 402 may be affixed to the femoral head/neck junction 406. In some embodiments, the tracking marker 402 is attached to an interface device 410. The interface device 410 may attach the tracking marker 402 to an affixation device 404. The interface device 410 may be adjustable via a screw, clamp, hinge, ratchet, or other adjustable element. The adjustments of the interface device 410 may be configured to produce known relative geometries between the tracking marker 402 and the affixation device 404. The affixation device 404 may be rigidly affixed to the anatomy (e.g., at the femoral head/neck junction 406). In certain embodiments, the affixation device 404 is a femoral head remover. A femoral head remover may include a corkscrew portion configured to be rotated to embed the corkscrew portion into the anatomy (e.g., at the femoral head/neck junction 406). Alternatively, the affixation device 404 may be a pin or a screw.

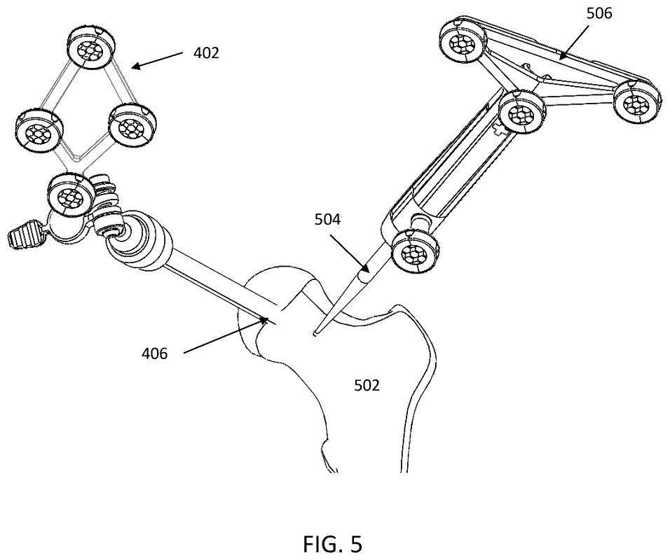

In some embodiments, the method 300 includes registering 304 the anatomy to the tracking marker 402. Registration may be performed relative to an image (e.g., a pre-operative or inter-operative CT, MRI, X-ray, fluorescent, ultrasound, etc.), a statistical shape model, and/or a patient-specific model based on some combination thereof. As illustrated in FIG. 5, registration 304 of the anatomy 502 may include capturing landmarks of the anatomy 502 using a point probe 504. The point probe 504 may be tracked, using an affixed probe tracking marker 506, to register the landmarks relative to the tracking marker 402.

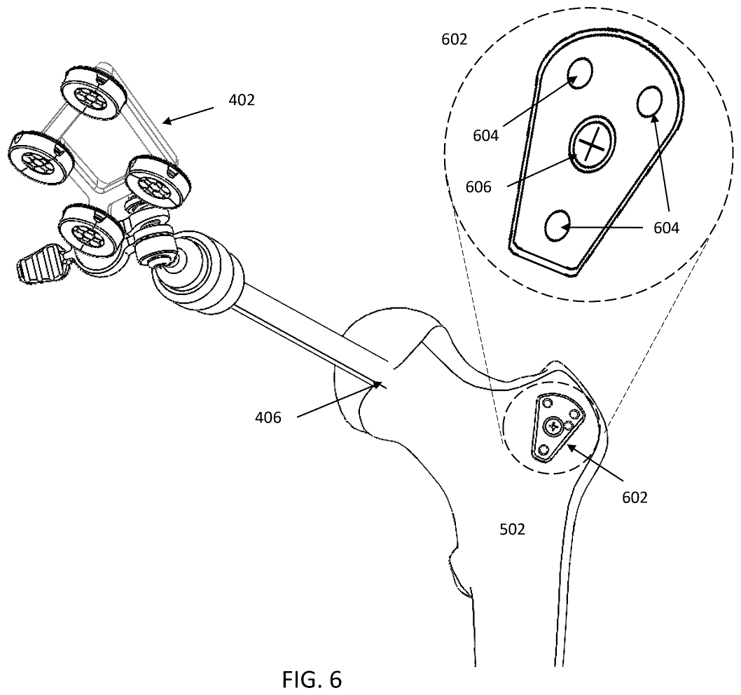

In certain embodiments, the method 300 includes attaching 306 a rigid, low-profile body (e.g., a plate) including at least three landmarks. FIG. 6 illustrates a rigid body 602, including landmarks 604, attached to the anatomy 502, in accordance with an embodiment. The landmarks 604 may be configured to be reliably captured by a point probe (e.g., point probe 504). The landmarks 604 may be divots or protrusions. In some embodiments, the landmarks 604 are arranged in a unique orientation relative to one another. The unique orientation may reduce ambiguity in determining an orientation/position of the rigid body 602 when collecting points using the point probe. Example unique orientations of the landmarks 604 may include a triangle, with each side being a distinct length. Alternatively, the triangle may include sides of the same or similar length (e.g., an isosceles triangle), with a unique orientation determinable based on assumptions associated with the data collection of the point using the point probe. For example, the system may assume the point probe is not interfering with the anatomy to access divots and/or that the assumed normal vector from the landmarks is away from the anatomy (i.e., not into the anatomy). Thus, removing ambiguity associated with an orientation of the landmarks 604. In a preferred embodiment, at least three landmarks are utilized for tracking across the x, y, and z dimensions (i.e., achieve the full six degrees of freedom constraint).

The rigid body 602 may include one or more mounting elements 606 (e.g., a screw) configured to rigidly affix the rigid body 602 to the anatomy 502. In particular, when a portion of the anatomy 502 is to be resected, the rigid body 602 is affixed to a different portion of the anatomy 502 (that will remain after resection) such that the rigid body 602 may be used for tracking means.

Although the landmarks 604 illustrated herein are representative of individual points, alternative landmarks 604 may be a groove or geometric shape formed into to the surface of the rigid body 602. In some embodiments, the at least three points may be collected by following the groove or tracing the geometric shape with the point probe 504. Alternatively, the at least three points may be collected at the ends and/or angles in the groove or corners in the geometric shape.

In some embodiments, the rigid body may be omitted entirely. In some embodiments, the landmarks 604 (e.g., divots, protrusions, grooves, and/or geometric shapes) may be directly applied to the anatomy 502. The landmarks 604 may include a deformation (e.g., to create a divot, groove, or geometric shape) on the surface of the anatomy 406 using a surgical tool (e.g., a drill or a cautery system). In alternative embodiments, the landmarks 604 may be distinct elements separately affixed or adhered to the anatomy 502. The landmarks 604 may be applied free hand, using a guide template, or through navigation with the tracking system 115.

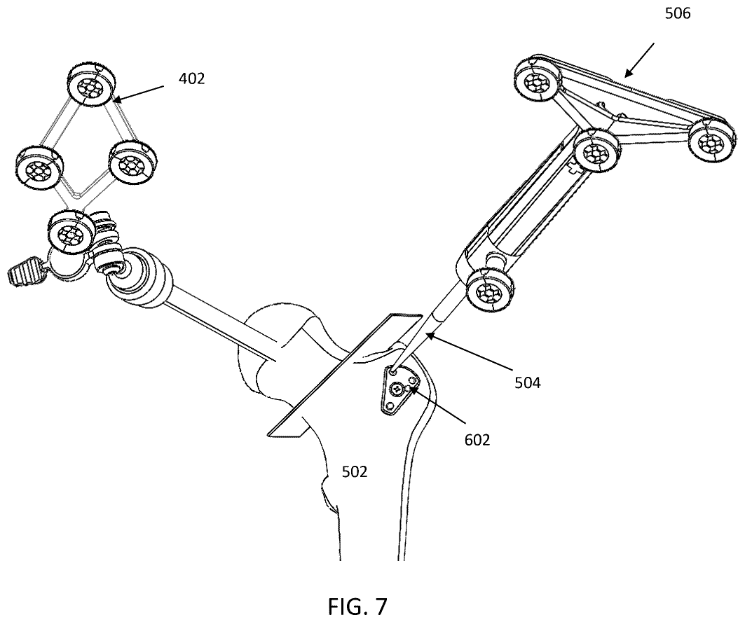

The method may include registering 308 these landmarks 604, and therefore the rigid body 602, relative to the tracking marker 402. Based on the prior registration between the tracking marker 402 and the anatomy 502, the landmarks 604 and the rigid body 602, may be registered with respect to the anatomy 502 as well. FIG. 7 illustrates collecting registration data from the landmarks 604 using a point probe 504 in accordance with an embodiment.

In some embodiments, the landmarks 604 may be used as a stable reference to display actual measurements during trialing (i.e., leg length/offset discrepancies) relative to the anatomy (e.g., the pelvis).

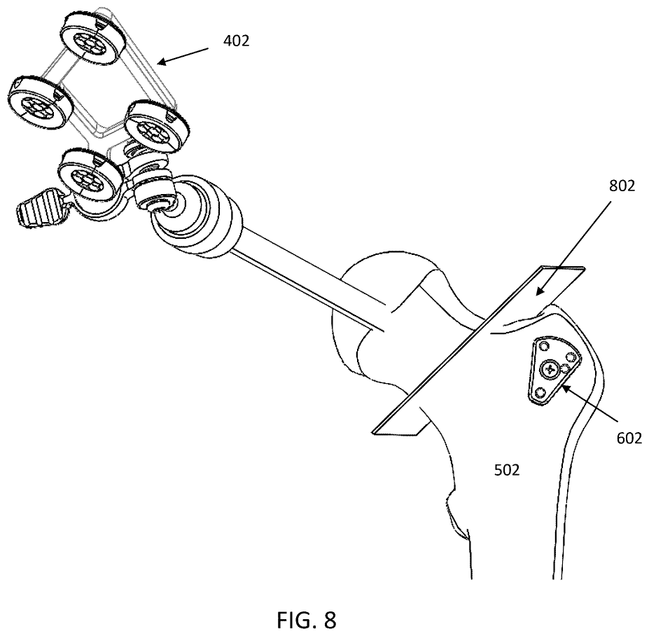

The method 300 may include removing 310 the tracking marker 402 from the anatomy 502. In some embodiments the tracking marker 402 may be removed from the anatomy 502, either by decoupling the tracking marker 402 at the interface device 410 or removing the affixation device 404 from the anatomy 502. In other embodiments, the anatomy 502 may be resected, removing the portion of the anatomy 502 to which the tracking marker 402 is affixed. FIG. 8 illustrates a cut plane 802 for a femoral neck cut, in accordance with an embodiment. In the example embodiment, the femoral neck cut removes the femoral head from the anatomy 502 (i.e., the femur), but leaves the rigid body 602 in place.

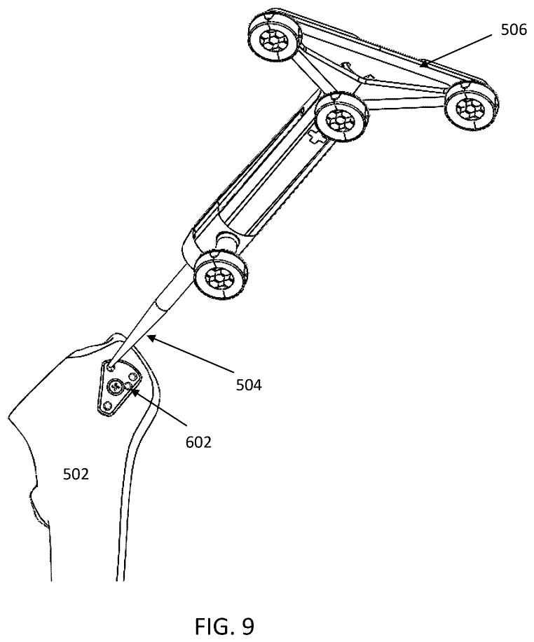

In some embodiments, the method 300 includes recollecting 312 the landmarks 604 with the anatomy 502 in a stable position. In the absence of the tracking marker 402, the position and/or orientation of the anatomy 502 may be recalled by recollecting the landmarks 604, giving a snapshot measurement of the anatomy in six degrees of freedom. As long as the anatomy 502 remains in a stable position, the snapshot measurement may be used. FIG. 9 illustrates acquiring a snapshot measurement of the anatomy 502 with a point probe 504 in accordance with an embodiment.

When collecting the landmark locations with a point probe 504, the landmark 604 positions may be defined as:

### DIVOTSTANATOMY=DIVOTSTSTABLE_ARRAY*STABLE_ARRAYTANATOMY

where DIVOTSTSTABLE_ARRAY is an affine transformation matrix comprising the spatial rotation, translation, scale changes, and affine transformations between the landmarks 604 and tracking marker 402, and STABLE_ARRAYTANATOMY is an affine transformation matrix comprising the spatial rotation, translation, scale changes, and affine transformations between the tracking marker 402 and the anatomy 502.

In the global reference frame (i.e., relative to the tracking system 115), the position of the anatomy 502 may be defined as:
CAMERATANATOMY=CAMERATPROBE_ARRAY*PROBE_ARRAY TPROBE_TIP*PROBE_TIPTDIVOTS*DIVOTSTSTABLE_ARRAY*STABLE_ARRAYTANATOMY
where CAMERATPROBE_ARRAY is an affine transformation matrix comprising the spatial rotation, translation, scale changes, and affine transformations between the tracking system 115 (e.g., a camera) and the probe tracking marker 506, PROBE_ARRAY TPROBE_TIP is an affine transformation matrix comprising the spatial rotation, translation, scale changes, and affine transformations between the probe tracking marker 506 and the probe 504 tip (i.e., the collection point), and PROBE_TIPTDIVOTS is an affine transformation matrix comprising the spatial rotation, translation, scale changes, and affine transformations between the probe 504 tip and the landmarks 602.

The transformation matrices may be based on collected information or known information (e.g., the geometry of point probe 504, tracking markers 402/506, or patient anatomy 502).

The transformation matrices may be used to define the position of the

anatomy 502 of interest in six degrees of freedom relative to any other tracked anatomy in the same reference space.

The method 300 may be used in conjunction with an operative plan (i.e., pre-operative and/or intraoperative) to define anticipated implant placement relative to the anatomy. For example, the CASS 100 may provide projected implant measurements (e.g., size, geometry, position, and/or orientation). The projected implant measurements may be used to control robotically assisted surgical devices to aid in completion of the procedure.

The method 300 allowing for the tracking of the anatomy 502 after removal of the tracking marker 402 may offer several benefits. For example, procedures performed according to the method 300 may allow six degrees of freedom measurement while eliminating stability concerns associated with large lever arms (e.g., affixation devices 404) attached to a small baseplate. The procedure may be improved by providing flexibility in tracking marker positioning while taking intraoperative measurements by not constraining the tracking marker in six degrees of freedom. Furthermore, the procedure may be improved by improving access by adding flexibility to the angle of approach. Additionally, the tracking marker may be simplified, necessitating fewer pins for stability as the tracking marker may be removed during mobile portions of the procedure. In addition, including fewer pins may reduce the likelihood of surgical complications.

The present disclosure describes a rigid plate attached to the femur in the area remaining after the femoral head is resected. This plate features three divots that facilitate tracking the position and orientation of the femur. This capability allows for the precise recall of the anatomical position and orientation of the patient. In surgical practice, this is referred to as leg length and offset measurement, which quantifies the distal and lateral movement of the femur. Surgeons typically compare these measurements to three reference points: the baseline state at the beginning of the operation, the preoperative plan, and the non-operative side.

One significant advantage of this plate is its role in anatomical registration. Many conventional systems require the leg to be placed in a “neutral” position before any surgical cuts are made, and again when recollecting data postoperatively. However, discrepancies between baseline and postoperative collections, such as a 5° misalignment, can lead to substantial measurement errors. For instance, a 5° abduction of the femur compared to the baseline can introduce a significant 5 mm error in leg length measurement, particularly at the greater trochanter position. Therefore, accurately restoring the femur to its preoperative position is crucial for precise leg length and offset measurements.

The three divots on the plate allow for virtual realignment of the femur to its preoperative state once the joint is reduced. “Reduction” in this context means restoring the femoral head to its correct anatomical position within the acetabulum, which is useful for maintaining joint stability and function. This ensures that there is no error due to femoral misalignment. Consequently, one of the primary benefits observed by surgeons using this workflow is that the calculation method is not sensitive to errors arising from femoral misalignment.

The process of determining leg length and offset begins with registering the femur anatomy relative to a tracking array. In the initial version of the software, this registration is based on a preoperative CT image, although it could also utilize MRI or an atlas-based model. The key step involves capturing the position of three divots on the femur relative to this frame, thereby establishing a reference for the anatomy.

During intraoperative leg length and offset measurements, the femoral head is reduced into the acetabular cup, and the three divots are collected. This allows for the determination of the femur position and orientation relative to the pelvis tracking array. When recollecting data postoperatively, the implant head is positioned in the implant cup center, and the three divots are collected again. This creates a coordinate system defined by the divots, which controls all six degrees of freedom—translational and rotational movements.

By realigning the axis of this coordinate system, the anatomical axis of the femur is effectively realigned. This ensures that the preoperative and postoperative conditions are aligned, with any differences in the offset and leg length directions corresponding to the surgical leg length and offset measurements. This method ensures that the femur is perfectly aligned with the preoperative collection, allowing for accurate and reliable measurements.

Although the systems and methods for tracking are disclosed relative to a THA, a person of ordinary skill in the art will understand that they may be adapted for other procedures on other anatomy (e.g., other joints). Furthermore, although in some embodiments the tracking markers are illustrated as optical tracking arrays, alternative tracking markers, as disclosed herein, are also considered.

### Femoral Neck Cut Guides for Navigated Surgery

During a THA procedure, surgeons may be required to make a cut on the femoral neck of a patient to remove the femoral head (e.g., as referenced with respect to FIG. 8) to replace the joint with implanted components. In a posterior approach, a single cut may be made to remove the femoral head. In a direct anterior approach, two cuts may be made to form a napkin ring that is removed prior to the rest of the femoral neck. The accuracy of these cuts may directly influence the positioning of the implanted femoral stem, which in turn impacts the leg length, caput-collum-diaphyseal angle, and offset. In a manual procedure, surgeons may rely on their own intuition and experience to determine where to cut the femoral neck. Surgeons lacking said experience may produce an inaccurate cut leading to errors between a planned fitting (e.g., using the CASS 100) and the actual surgical alignment.

While surgeons often manually perform this cut during a procedure. The surgeon may utilize a marking device, such as a pen or an electrosurgical device (i.e., a Bovie), to mark a planned location for the cut on the bone. The marking may help with maintaining a consistent resection level. In some embodiments, the marking device may be tracked (e.g., relative to the tracking marker 402) and guided (relative to the anatomy 502) to marker the bone at the proper resection level to further increase accuracy. To mark at the correct location using navigation, surgeons may be required to align themselves with the target anatomy in a display and trace along the bone while remaining at the correct resection level according to their navigation system. Alternatively, a guide may be utilized in tandem with navigation to precisely align the level and angle. However, utilizing a cut guide adds extra steps to the surgical procedure for mounting and adjusting the guide to the desired location.

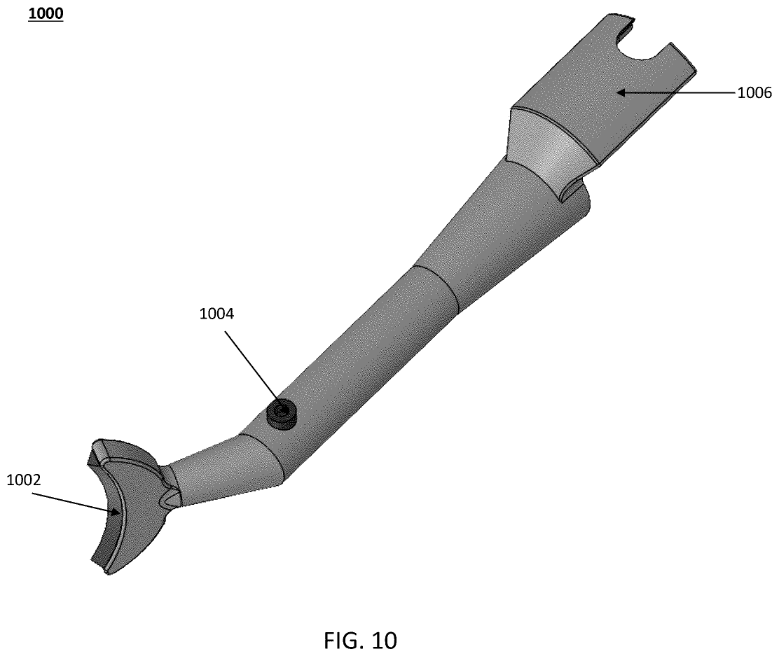

FIG. 10 depicts an electrosurgical device 1000 configured to mark the bone at a proper resection level and angle in accordance with an embodiment. The electrosurgical device 1000 may be tracked and guided according to a known position and/or orientation of a tracking marker or the femur. The electrosurgical device 1000 may be placed at the desired neck cut level where it may be activated to mark the bone.

In some embodiments, the electrosurgical device 1000 includes a connection point 1004 for a Bovie, or similar device, to be attached. The electrosurgical device 1000 may include a conductive femoral neck guide 1002 configured to contact with the bone.

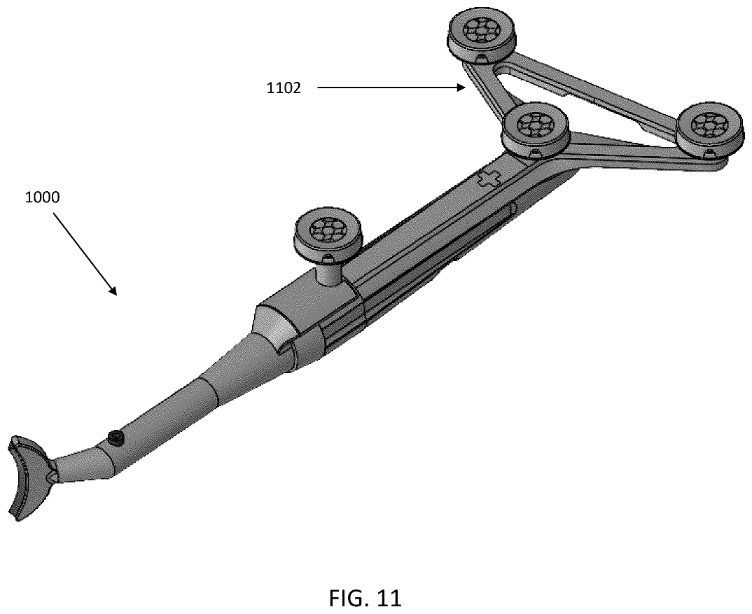

In certain embodiments, the electrosurgical device 1000 includes an interface 1006 for a tracking element. The tracking element may be a tracking marker or a tool including a tracking marker (e.g., a point probe). FIG. 11 depicts the electrosurgical device 1000 interfaced with a point probe 1102 in accordance with an embodiment. Interfacing the electrosurgical device 1000 with a tool including a tracking marker, such as a point probe, may limit excessive equipment cluttering the surgical space.

The electrosurgical device 1000 may be aligned, using navigation, to the preplanned neck cut level. The navigation may be aided by a display illustrating the current tracked position of the electrosurgical device 1000 (e.g., the current contact position on the anatomy) in comparison to a planned position. Once correctly positioned, a user may interact with the connection point 1004 to enable a flow of current to the conductive femoral neck guide 1002 in contact with the bone. In some embodiments, the Bovie may be used in a monopolar mode, which necessitates a secondary electrode to be attached to the body of the patient to complete a conductive circuit through them. With a waveform (e.g., a high current and low voltage AC waveform) applied to the Bovie, the femoral neck guide 1002 may desiccate the bone at the location of direct contact before the applied current disperses through the bone to the electrode. As a result, the bone may be seared in a line at the exact neck cut level. The seared line may be used to guide the removal of the femoral head.

### Data Processing Systems for Implementing Embodiments Herein

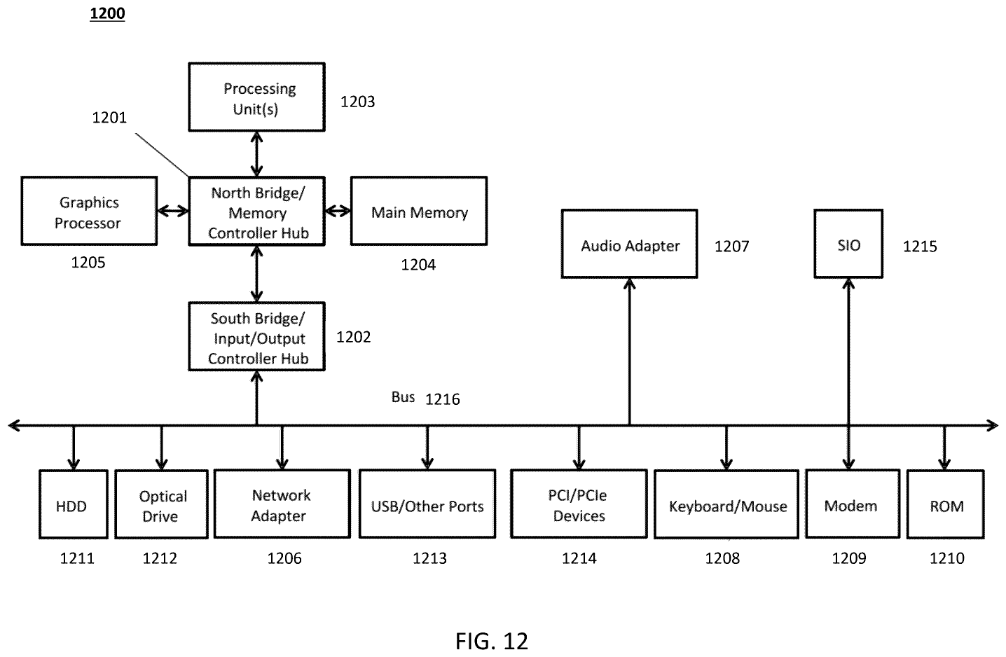

FIG. 12 illustrates a block diagram of an exemplary data processing system 1200 in which embodiments are implemented. The data processing system 1200 is an example of a computer, such as a server or client, in which computer usable code or instructions implementing the process for illustrative embodiments of the present invention are located. In some embodiments, the data processing system 1200 may be a server computing device. For example, the data processing system 1200 may be implemented in a server or another similar computing device operably connected to a surgical system 100 as described above. The data processing system 1200 may be configured to, for example, transmit and receive information related to a patient and/or a related surgical plan with the surgical system 100.

In the depicted example, the data processing system 1200 may employ a hub architecture including a north bridge and memory controller hub (NB/MCH) 1201 and south bridge and input/output (I/O) controller hub (SB/ICH) 1202. A processing unit 1203, a main memory 1204, and a graphics processor 1205 may be connected to the NB/MCH 1201. The graphics processor 1205 may be connected to the NB/MCH 1201 through, for example, an accelerated graphics port (AGP).

In the depicted example, a network adapter 1206 connects to the SB/ICH 1202. An audio adapter 1207, a keyboard and mouse adapter 1208, a modem 1209, a read only memory (ROM) 1210, a hard disk drive (HDD) 1211, an optical drive (e.g., CD or DVD) 1212, a universal serial bus (USB) ports and other communication ports 1213, and PCI/PCIe devices 1214 may connect to the SB/ICH 1202 through a bus system 1216. The PCI/PCIe devices 1214 may include Ethernet adapters, add-in cards, and/or PC cards for notebook computers. The ROM 1210 may be, for example, a flash basic input/output system (BIOS). The HDD 1211 and the optical drive 1212 may use an integrated drive electronics (IDE) or serial advanced technology attachment (SATA) interface. A super I/O (SIO) device 1215 may be connected to the SB/ICH 1202.

An operating system may run on the processing unit 1203. The operating system may coordinate and provide control of various components within the data processing system 1200. As a client, the operating system may be a commercially available operating system. An object-oriented programming system, such as the Java™ programming system, may run in conjunction with the operating system and provide calls to the operating system from the object-oriented programs or applications executing on the data processing system 1200. As a server, the data processing system 1200 may be an IBM® eServer™ System® running the Advanced Interactive Executive operating system or the Linux operating system. The data processing system 1200 may be a symmetric multiprocessor (SMP) system that includes a plurality of processors in the processing unit 1203. Alternatively, a single processor system may be employed.

Instructions for the operating system, the object-oriented programming system, and applications or programs are located on storage devices, such as the HDD 1211, and are loaded into the main memory 1204 for execution by the processing unit 1203. The processes for embodiments described herein may be performed by the processing unit 1203 using computer usable program code, which can be located in a memory such as, for example, main memory 1204, ROM 1210, or in one or more peripheral devices.

A bus system 1216 may comprise one or more busses. The bus system 1216 may be implemented using any type of communication fabric or architecture that provides for a transfer of data between different components or devices attached to the fabric or architecture. A communication unit such as the modem 1209 or the network adapter 1206 may include one or more devices that can be used to transmit and receive data.

Those of ordinary skill in the art will appreciate that the hardware depicted in FIG. 12 may vary depending on the implementation. Other internal hardware or peripheral devices, such as flash memory, equivalent non-volatile memory, or optical disk drives may be used in addition to or in place of the hardware depicted. Moreover, the data processing system 1200 can take the form of any of a number of different data processing systems, including but not limited to, client computing devices, server computing devices, tablet computers, laptop computers, telephone or other communication devices, personal digital assistants, and the like. Essentially, data processing system 1200 can be any known or later developed data processing system without architectural limitation.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices also can “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A tracking device comprising:
a rigid plate comprising one or more affixation points configured to receive a screw for securing the rigid plate to patient anatomy; and
at least three divots located on an outfacing surface of the rigid plate, wherein a location of each divot is configured to be reliably captured using a point probe.

2. The tracking device of claim 1, wherein the at least three divots are arranged on the outfacing surface in an at least partially asymmetrical configuration, such that an orientation of the rigid plate is unambiguous based on a location of the at least three divots.

3. An electrosurgical device comprising:
a housing;
an electrically conductive femoral neck guide configured to a shape of a femoral neck, wherein the electrically conductive femoral neck guide is rigidly affixable to the housing;
a control configured to apply a high current, low voltage AC waveform to the electrically conductive femoral neck guide; and
a tracking marker rigidly affixable to the housing.

4. The electrosurgical device of claim 3, wherein the tracking marker comprises a point probe, wherein the point probe is removable from the housing.

5. The electrosurgical device of claim 3, wherein the control is configured to apply the AC waveform in response to an input from a tracking system suggesting the electrically conductive femoral neck guide is placed according to a surgical plan.

6. The electrosurgical device of claim 5, wherein the control is configured to apply the AC waveform in further response to a manual input.

7. A computer-implemented method comprising:
receiving, by a processor, at least one of an image and a statistical shape model of a patient anatomy;
acquiring, using a tracking system in communication with the processor, a tracking marker location of a tracking marker;
acquiring, using the tracking system, a plurality of point probe locations of a trackable point probe, wherein for each of the plurality of point probe locations the trackable point probe is positioned relative to the patient anatomy;
registering, by the processor, a first position and orientation of the patient anatomy relative to the tracking marker based on the tracking marker location and the plurality of point probe locations;
acquiring, using the tracking system, landmark locations of at least three landmarks on a rigid, low-profile body configured to affix to the patient anatomy;
registering, by the processor, a position and orientation of the rigid, low-profile body relative to the tracking marker location based on the tracking marker location and the landmark locations;
generating, by the processor, a transformation matrix between the position and orientation of the rigid, low-profile body and the first position and orientation of the patient anatomy;
acquiring, using the tracking system, updated landmark locations of the at least three landmarks, absent the tracking marker; and
determining, by the processor, a second position and orientation of the patient anatomy based on the updated landmark locations and the transformation matrix.

8. The computer-implemented method of claim 7, wherein each of the at least three landmarks comprises a divot configured to be reliably captured by the trackable point probe.

9. The computer-implemented method of claim 7, wherein the at least three landmarks are arranged on a surface of the rigid, low-profile body in an at least partially asymmetrical configuration, such that an orientation of the rigid, low-profile body is unambiguous based on a location of the at least three landmarks.

10. The computer-implemented method of claim 7, further comprising:
navigating, by the processor, a marking device configured to mark the patient anatomy for a cut between a first affixation point of the tracking marker to the patient anatomy and a second affixation point of the rigid, low-profile body to the patient anatomy.

11. The computer-implemented method of claim 10, wherein the cut is configured to remove a femoral head from the patient anatomy, and wherein the tracking marker is affixed to the femoral head.

12. The computer-implemented method of claim 7, wherein the tracking marker is affixed to the patient anatomy via a femoral head remover.

13. The computer-implemented method of claim 7, wherein the tracking system is an optical tracking system, the method further comprising acquiring, using the tracking system, the position and orientation of the rigid, low-profile body based on an optical detection of the landmark locations.

14. A system comprising:
a tracking system;
a tracking marker configured to affix to a patient anatomy at a first affixation point;
a trackable point probe;
a rigid, low-profile body configured to affix to the patient anatomy at a second affixation point, wherein the rigid, low-profile body comprises at least three landmarks;
a processor in communication with the tracking system; and
a non-transitory, processor-readable storage medium, wherein the non-transitory, processor-readable storage medium comprises one or more programming instructions that, when executed, cause the processor to:
receive at least one of an image and a statistical shape model of the patient anatomy;
acquire, using the tracking system, a tracking marker location of the tracking marker;
acquire, using the tracking system, a plurality of point probe locations of the trackable point probe, wherein for each of the plurality of point probe locations the trackable point probe is positioned relative to the patient anatomy;
register a first position and orientation of the patient anatomy relative to the tracking marker based on the tracking marker location and the plurality of point probe locations;
acquire, using the tracking system, landmark locations of the at least three landmarks;
register a position and orientation of the rigid, low-profile body relative to the tracking marker location based on the tracking marker location and the landmark locations;
generate a transformation matrix between the position and orientation of the rigid, low-profile body and the first position and orientation of the patient anatomy;
acquire, using the tracking system, updated landmark locations of the at least three landmarks, absent the tracking marker; and
determine a second position and orientation of the patient anatomy based on the updated landmark locations and the transformation matrix.

15. The system of claim 14, wherein each of the at least three landmarks comprises a divot configured to be reliably captured by the trackable point probe.

16. The system of claim 14, wherein the at least three landmarks are arranged on a surface of the rigid, low-profile body in an at least partially asymmetrical configuration, such that an orientation of the rigid, low-profile body is unambiguous based on a location of the at least three landmarks.

17. The system of claim 14, further comprising a marking device, wherein the one or more programming instructions further cause the processor to navigate the marking device configured to mark the patient anatomy for a cut between a first affixation point of the tracking marker to the patient anatomy and a second affixation point of the rigid, low-profile body to the patient anatomy.

18. The system of claim 17, wherein the marking device is an electrosurgical device.

19. The system of claim 17, wherein the cut is configured to remove a femoral head from the patient anatomy, and wherein the tracking marker is affixed to the femoral head.

20. The system of claim 17, wherein the tracking marker is affixed to the patient anatomy via a femoral head remover.
